(19) United States
(12) Patent Application Publication
(10) Pub. No.: US 2025/0262259 A1
(43) Pub. Date: Aug. 21, 2025

(54) GEL FOR TREATING HEMORRHOIDS AND PREPARATION METHOD THEREOF

(71) Applicant: Changde Jizhi Biological Technology Co., Ltd, Changde (CN)

(72) Inventor: Xinqiao HE, Changde (CN)

(21) Appl. No.: 18/582,796

(22) Filed: Feb. 21, 2024

(51) Int. Cl. A61K 36/288; A61K 9/06; A61K 36/062; A61K 36/16; A61K 36/258; A61K 36/28; A61K 36/489; A61K 36/51; A61K 36/739; A61K 36/82; A61K 47/10; A61K 47/36

(52) U.S. Cl. CPC A61K 36/288; A61K 9/06; A61K 36/062; A61K 36/16; A61K 36/258; A61K 36/28; A61K 36/489; A61K 36/51; A61K 36/739; A61K 36/82; A61K 47/10; A61K 47/36; A61K 2236/15; A61K 2236/33

(57) ABSTRACT

A gel for treating hemorrhoids and a preparation method thereof, related to the field of medical technologies, are provided. The gel includes Taraxacum mongolicum, Gerbera jamesonii bolus, Panax notoginseng, Monascus purpureus went, Sanguisorba officinalis, chitosan, Camellia oleifera abel, glycerine, Ginkgo biloba, Swertia manshurica and Sophora japonica. The gel can exert significant synergistic effects, have good therapeutic effects, and high safety, which is of great significance for the prevention and treatment of hemorrhoids.

### TECHNICAL FIELD

The disclosure relates to the field of medical technologies, and particularly to a gel for treating hemorrhoids and a preparation method thereof.

### BACKGROUND

Hemorrhoids (commonly known as piles) are a common disease located in the anal area that can develop at any age, but the incidence gradually increases with age. According to the latest epidemiological survey data, the prevalence of hemorrhoids is as high as 50%.

There are two main theories regarding the etiology of hemorrhoids. The first is the varices theory, which holds that hemorrhoids are venous mass formed by stasis, dilatation and flexion of the venous plexus under the mucous membranes of the lower part of the rectum and under the skin of the anal canal. However, the widely accepted theory currently is Thomson's theory of anal cushion shift-down, which suggests that hemorrhoids are originally a normal anatomical structure of the anal canal, namely a vascular cushion, which is a circular sponge like tissue band 1.5 cm above the dentate line. Only when the anal cushion tissue is abnormal and combined with symptoms, the anal cushion tissue can be called hemorrhoids, and need to be treated. The purpose of the treatment is to relieve the symptoms, not eliminate the hemorrhoid body. There are many triggering factors for hemorrhoids, among which constipation, prolonged drinking, eating a lot of stimulating food and sitting for a long time are the main triggers. Common symptoms of hemorrhoids are hematochezia, a feeling of swelling and pain, the occurrence of thrombosis and inflammation, and in severe cases, progression to prolapse.

The specific pathogenesis of hemorrhoids is not yet fully understood and may be related to multiple factors. The theory of varicose veins suggests that its pathogenesis is that the venous plexus is the main structure that forms the anal cushion, and the formation of hemorrhoids is inevitably related to the pathological dilation and thrombosis of the venous plexus. Anatomically speaking, the portal vein system and its branches of the rectal veins do not have venous valves, the walls of the upper and lower rectal venous plexus are thin and shallow in location, and relaxation of submucosal tissue in the distal rectum can easily lead to blood stasis and venous dilation. In addition, due to the fact that the rectal and anal canal is located at the bottom of the abdominal cavity, various factors such as long-term sitting, constipation, pregnancy, prostate enlargement, and huge pelvic tumors can cause venous flow obstruction of rectum. The theory of anal cushion shift-down suggests that the anal cushion plays a role in closing the anal canal and regulating bowel movements. Under normal circumstances, the anal pad loosely adheres to the anal muscle wall. When defecating, the anal cushion is pushed down by downward pressure, and after defecating, the anal cushion uses its own contraction to retract back into the anal canal. After the clastic retraction ability weakens, the anal cushion becomes congested and down shift to form hemorrhoids.

At present, the treatment of hemorrhoids mainly uses medicines or surgery to treat hemorrhoids. However, many people have not reached the indications for surgery, and patients who want conservative treatment suffer from the lack of the medicines available. Patients live in extreme fear and pain every day, and people are also conducting related research.

### SUMMARY

The main purpose of the disclosure is to provide a gel for treating hemorrhoids and a preparation method thereof. The gel of the disclosure has good therapeutic effect and high safety, which is of great significance for the prevention and treatment of hemorrhoids.

In order to achieve above purpose, the technical solutions are as follows.

A gel for treating hemorrhoids and a preparation method thereof are provided, the gel includes Taraxacum mongolicum, Gerbera jamesonii bolus, Panax notoginseng, Monascus purpureus went, Sanguisorba officinalis, chitosan, Camellia oleifera abel, glycerine, Ginkgo biloba, Swertia manshurica and Sophora japonica. The Taraxacum mongolicum is 20-80 parts by weight, the Gerbera jamesonii bolus is 20-80 parts by weight, the Panax notoginseng is 50-150 parts by weight, the Monascus purpureus went is 50-150 parts by weight, the Sanguisorba officinalis is 10-50 parts by weight, the chitosan is 10-40 parts by weight, the Camellia oleifera abel is 5-25 parts by weight, the glycerine is 5-25 parts by weight, the Ginkgo biloba is 10-40 parts by weight, the Swertia manshurica is 10-50 parts by weight and the Sophora japonica is 10-50 parts by weight.

In an embodiment, the Taraxacum mongolicum is 20 parts by weight, the Gerbera jamesonii bolus is 20 parts by weight, the Panax notoginseng is 50 parts by weight, the Monascus purpureus went is 50 parts by weight, the Sanguisorba officinalis is 10 parts by weight, the chitosan is 10 parts by weight, the Camellia oleifera abel is 5 parts by weight, the glycerine is 5 parts by weight, the Ginkgo biloba is 10 parts by weight, the Swertia manshurica is 10 parts by weight and the Sophora japonica is 10 parts by weight.

In an embodiment, the Taraxacum mongolicum is 50 parts by weight, the Gerbera jamesonii bolus is 50 parts by weight, the Panax notoginseng is 100 parts by weight, the Monascus purpureus went is 100 parts by weight, the Sanguisorba officinalis is 30 parts by weight, the chitosan is 25 parts by weight, the Camellia oleifera abel is 15 parts by weight, the glycerine is 15 parts by weight, the Ginkgo biloba is 25 parts by weight, the Swertia manshurica is 30 parts by weight and the Sophora japonica is 30 parts by weight.

A weight ratio of the Taraxacum mongolicum, the Gerbera jamesonii bolus, the Panax notoginseng, the Monascus purpureus went, the Sanguisorba officinalis, the Ginkgo biloba, the Swertia manshurica, and the Sophora japonica is 1:1:1.5-3:1.5-3:0.4-1:0.4-0.8:0.4-1:0.4-1; and a weight ratio of the Taraxacum mongolicum to the chitosan is 1.5-3:1.

In an embodiment, the Taraxacum mongolicum is 80 parts by weight, the Gerbera jamesonii bolus is 80 parts by weight, the Panax notoginseng is 150 parts by weight, the Monascus purpureus went is 150 parts by weight, the Sanguisorba officinalis is 50 parts by weight, the chitosan is 40 parts by weight, the Camellia oleifera abel is 25 parts by weight, the glycerine is 25 parts by weight, the Ginkgo biloba is 40 parts by weight, the Swertia manshurica is 50 parts by weight and the Sophora japonica is 50 parts by weight.

In an embodiment, the preparation method includes the following steps:
(1) the Taraxacum mongolicum, the Gerbera jamesonii bolus, the Panax notoginseng, the Monascus purpureus went, the Sanguisorba officinalis, the Ginkgo biloba, the Swertia manshurica, and the Sophora japonica are taken according to the parts by weight, and the Taraxacum mongolicum, the Gerbera jamesonii bolus, the Panax notoginseng, the Monascus purpureus went, the Sanguisorba officinalis, the Ginkgo biloba, the Swertia manshurica, and the Sophora japonica are crushed thoroughly in a grinder, followed by sieving to obtain a Taraxacum mongolicum powder, a Gerbera jamesonii bolus powder, a Panax notoginseng powder, a Monascus purpureus went powder, a Sanguisorba officinalis powder, a Ginkgo biloba powder, a Swertia manshurica powder, and a Sophora japonica powder, respectively;
(2) an ethanol heating reflux extraction is utilized to obtain a Taraxacum mongolicum ethanol extract, a Gerbera jamesonii bolus ethanol extract, a Panax notoginseng ethanol extract, a Monascus purpureus went ethanol extract, a Sanguisorba officinalis ethanol extract, a Ginkgo biloba ethanol extract, a Swertia manshurica ethanol extract, and a Sophora japonica ethanol extract from the Taraxacum mongolicum powder, the Gerbera jamesonii bolus powder, the Panax notoginseng powder, the Monascus purpureus went powder, the Sanguisorba officinalis powder, the Ginkgo biloba powder, the Swertia manshurica powder and the Sophora japonica powder obtained from step (1); and
(3) the ethanol extracts obtained in step (2), the chitosan, the Camellia oleifera abel and the glycerine are mixed, and then stirred evenly to obtain the gel for treating hemorrhoids.

In an embodiment, the step (2) includes: performing the following steps for each of the Taraxacum mongolicum powder, the Gerbera jamesonii bolus powder, the Panax notoginseng powder, the Monascus purpureus went powder, the Sanguisorba officinalis powder, the Ginkgo biloba powder, the Swertia manshurica powder and the Sophora japonica powder: heating reflux extraction is performed on the powder with 85-90% ethanol for 1-3 times, followed by recovering a solvent under a reduced pressure to obtain an extract, then the extract is dispersed in a distilled water, and extracted for 2-4 times with ethyl acetate to obtain the ethanol extract.

In an embodiment, the step (2) includes: performing the following steps for each of the Taraxacum mongolicum powder, the Gerbera jamesonii bolus powder, the Panax notoginseng powder, the Monascus purpureus went powder, the Sanguisorba officinalis powder, the Ginkgo biloba powder, the Swertia manshurica powder and the Sophora japonica powder: heating reflux extraction is performed on the powder with 85% ethanol for 3 times, followed by recovering a solvent under a reduced pressure to obtain an extract, then the extract is dispersed in a distilled water, and extracted for 3 times with ethyl acetate to obtain the ethanol extract.

Compared with the prior art, the beneficial effects of the disclosure are as follows.

A gel for treating hemorrhoids and a preparation method thereof are provided, the components in the gel for treating hemorrhoids are compatible and optimized by adjusting a ratio of Taraxacum mongolicum, gerbera jamesonii bolus, Panax notoginseng, Monascus purpureus went, and Sanguisorba officinalis, and a ratio of Taraxacum mongolicum to the chitosan. The gel can play an obvious synergistic role, which has a significant role in clearing heat, relieving pain, promoting blood circulation, removing blood stasis, swelling, and promoting muscle growth. The gel can be further used for internal and external hemorrhoids caused by various factors. Moreover, the gel has the advantages of natural, safe, and mild efficacy, non-toxic side effects, simple preparation method, low cost, and conducive to large-scale production.

### DETAILED DESCRIPTION OF EMBODIMENTS

In order to facilitate a better understanding of the disclosure by those skilled in the art, the following is a clear and complete description of the technical solutions in the embodiments of the disclosure. Apparently, the described embodiments are only a part of the embodiments of the disclosure, not all of them. Based on the embodiments in the disclosure, all other embodiments obtained by those skilled in the art without creative labor shall fall within the scope of protection of the disclosure.

### Embodiment 1

A gel for treating hemorrhoids and a preparation method thereof are provided, the gel includes: 20 parts by weight of Taraxacum mongolicum, 20 parts by weight of Gerbera jamesonii bolus, 50 parts by weight of Panax notoginseng, 50 parts by weight of Monascus purpureus went, 10 parts by weight of Sanguisorba officinalis, 10 parts by weight of chitosan, 5 parts by weight of Camellia oleifera abel, 5 parts by weight of glycerine, 10 parts by weight of Ginkgo biloba, 10 parts by weight of Swertia manshurica and 10 parts by weight of Sophora japonica.

The preparation method includes the following steps: (1) the Taraxacum mongolicum, the Gerbera jamesonii bolus, the Panax notoginseng, the Monascus purpureus went, the Sanguisorba officinalis, the Ginkgo biloba, the Swertia manshurica, and the Sophora japonica according to the parts by weight are taken to crush thoroughly in a grinder, followed by sieving to obtain a Taraxacum mongolicum powder, a Gerbera jamesonii bolus powder, a Panax notoginseng powder, a Monascus purpureus went powder, a Sanguisorba officinalis powder, a Ginkgo biloba powder, a Swertia manshurica powder, and a Sophora japonica powder, respectively; (2) an ethanol heating reflux extraction is utilized to obtain a Taraxacum mongolicum ethanol extract, a Gerbera jamesonii bolus ethanol extract, a Panax notoginseng ethanol extract, a Monascus purpureus went ethanol extract, a Sanguisorba officinalis ethanol extract, a Ginkgo biloba ethanol extract, a Swertia manshurica ethanol extract, and a Sophora japonica ethanol extract from the Taraxacum mongolicum powder, the Gerbera jamesonii bolus powder, the Panax notoginseng powder, the Monascus purpureus went powder, the Sanguisorba officinalis powder, the Ginkgo biloba powder, the Swertia manshurica powder and the Sophora japonica powder obtained from step (1); (3) the ethanol extracts obtained in step (2), the chitosan, the Camellia oleifera abel and the glycerine are mixed, and then stirred evenly according to the parts by weight to obtain the gel for treating hemorrhoids.

In an embodiment, a preparation method of each ethanol extract in step (2) includes the following steps: the heating reflux extraction is performed on the powder with 90% ethanol for 1 time, followed by recovering a solvent under a reduced pressure to obtain an extract, then the extract is dispersed in a distilled water, and extracted for 2 times with ethyl acetate to obtain the ethanol extract.

In an embodiment, a preparation method of each ethanol extract in step (2) includes the following steps: the heating reflux extraction is performed on the powder with 85% ethanol for 3 times, followed by recovering a solvent under an reduced pressure to obtain an extract, then the extract is dispersed in a distilled water, and extracted for 3 times with the ethyl acetate to obtain the ethanol extract.

### Embodiment 2

A gel for treating hemorrhoids and a preparation method thereof are provided. The gel includes: 50 parts by weight of Taraxacum mongolicum, 50 parts by weight of Gerbera jamesonii bolus, 100 parts by weight of Panax notoginseng, 100 parts by weight of Monascus purpureus went, 30 parts by weight of Sanguisorba officinalis, 25 parts by weight of the chitosan, 15 parts by weight of Camellia oleifera abel, 15 parts by weight of glycerine, 25 parts by weight of Ginkgo biloba, 30 parts by weight of Swertia manshurica and 30 parts by weight of Sophora japonica.

The preparation method includes the following steps: (1) the Taraxacum mongolicum, the Gerbera jamesonii bolus, the Panax notoginseng, the Monascus purpureus went, the Sanguisorba officinalis, the Ginkgo biloba, the Swertia manshurica, and the Sophora japonica according to the parts by weight are taken to crush thoroughly in a grinder, followed by sieving to obtain a Taraxacum mongolicum powder, a Gerbera jamesonii bolus powder, a Panax notoginseng powder, a Monascus purpureus went powder, a Sanguisorba officinalis powder, a Ginkgo biloba powder, a Swertia manshurica powder, and a Sophora japonica powder, respectively; (2) an ethanol heating reflux extraction is utilized to obtain a Taraxacum mongolicum ethanol extract, a Gerbera jamesonii bolus ethanol extract, a Panax notoginseng ethanol extract, a Monascus purpureus went ethanol extract, a Sanguisorba officinalis ethanol extract, a Ginkgo biloba ethanol extract, a Swertia manshurica ethanol extract, and a Sophora japonica ethanol extract from the Taraxacum mongolicum powder, the Gerbera jamesonii bolus powder, the Panax notoginseng powder, the Monascus purpureus went powder, the Sanguisorba officinalis powder, the Ginkgo biloba powder, the Swertia manshurica powder and the Sophora japonica powder obtained from step (1); (3) the ethanol extracts obtained in step (2), the chitosan, the Camellia oleifera abel and the glycerine are mixed, and then stirred evenly according to the parts by weight to obtain the gel for treating hemorrhoids.

In an embodiment, a preparation method of each ethanol extract in step (2) includes the following steps: the heating reflux extraction is performed on the powder with 85% ethanol for 2 times, followed by recovering a solvent under a reduced pressure to obtain an extract, then the extract is dispersed in a distilled water, and extracted for 3 times with ethyl acetate to obtain the ethanol extract.

In an embodiment, a preparation method of each ethanol extract in step (2) includes the following steps: the heating reflux extraction is performed on the powder with 85% ethanol for 3 times, followed by recovering a solvent under a reduced pressure to obtain an extract, then the extract is dispersed in a distilled water, and extracted for 3 times with ethyl acetate to obtain the ethanol extract.

### Embodiment 3

A gel for treating hemorrhoids and a preparation method thereof are provided. The gel includes: 80 parts by weight of Taraxacum mongolicum, 80 parts by weight of Gerbera jamesonii bolus, 150 parts by weight of Panax notoginseng, 150 parts by weight of Monascus purpureus went, 50 parts by weight of Sanguisorba officinalis, 40 parts by weight of the chitosan, 25 parts by weight of Camellia oleifera abel, 25 parts by weight of the glycerine, 40 parts by weight of Ginkgo biloba, 50 parts by weight of Swertia manshurica and 50 parts by weight of Sophora japonica.

The preparation method includes the following steps: (1) the Taraxacum mongolicum, the Gerbera jamesonii bolus, the Panax notoginseng, the Monascus purpureus went, the Sanguisorba officinalis, the Ginkgo biloba, the Swertia manshurica, and the Sophora japonica according to the parts by weight are taken to crush thoroughly in a grinder, followed by sieving to obtain a Taraxacum mongolicum powder, a Gerbera jamesonii bolus powder, a Panax notoginseng powder, a Monascus purpureus went powder, a Sanguisorba officinalis powder, a Ginkgo biloba powder, a Swertia manshurica powder, and a Sophora japonica powder, respectively; (2) an ethanol heating reflux extraction is utilized to obtain a Taraxacum mongolicum ethanol extract, a Gerbera jamesonii bolus ethanol extract, a Panax notoginseng ethanol extract, a Monascus purpureus went ethanol extract, a Sanguisorba officinalis ethanol extract, a Ginkgo biloba ethanol extract, a Swertia manshurica ethanol extract, and a Sophora japonica ethanol extract from the Taraxacum mongolicum powder, the Gerbera jamesonii bolus powder, the Panax notoginseng powder, the Monascus purpureus went powder, the Sanguisorba officinalis powder, the Ginkgo biloba powder, the Swertia manshurica powder and the Sophora japonica powder obtained from step (1); (3) the ethanol extracts obtained in step (2), the chitosan, the Camellia oleifera abel and the glycerine are mixed, and then stirred evenly according to the parts by weight to obtain the gel for treating hemorrhoids.

In an embodiment, a preparation method of each ethanol extract in step (2) includes the following steps: the heating reflux extraction is performed on the powder with 90% ethanol for 3 times, followed by recovering a solvent under a reduced pressure to obtain an extract, then the extract is dispersed in a distilled water, and extracted for 4 times with ethyl acetate to obtain the ethanol extract.

In an embodiment, a preparation method of each ethanol extract in step (2) includes the following steps: the heating reflux extraction is performed on the powder with 85% ethanol for 3 times, followed by recovering a solvent under an reduced pressure to obtain an extract, then the extract is dispersed in a distilled water, and extracted for 3 times with the ethyl acetate to obtain the ethanol extract.

### Embodiment 4

A gel for treating hemorrhoids and a preparation method thereof are provided. The gel includes: 30 parts by weight of Taraxacum mongolicum, 30 parts by weight of Gerbera jamesonii bolus, 70 parts by weight of Panax notoginseng, 70 parts by weight of Monascus purpureus went, 15 parts by weight of Sanguisorba officinalis, 15 parts by weight of the chitosan, 10 parts by weight of Camellia oleifera abel, 10 parts by weight of the glycerine, 15 parts by weight of Ginkgo biloba, 15 parts by weight of Swertia manshurica and 15 parts by weight of Sophora japonica.

The preparation method includes the following steps: (1) the Taraxacum mongolicum, the Gerbera jamesonii bolus, the Panax notoginseng, the Monascus purpureus went, the Sanguisorba officinalis, the Ginkgo biloba, the Swertia manshurica, and the Sophora japonica according to the parts by weight are taken to crush thoroughly in a grinder, followed by sieving to obtain a Taraxacum mongolicum powder, a Gerbera jamesonii bolus powder, a Panax notoginseng powder, a Monascus purpureus went powder, a Sanguisorba officinalis powder, a Ginkgo biloba powder, a Swertia manshurica powder, and a Sophora japonica powder, respectively; (2) an ethanol heating reflux extraction is utilized to obtain a Taraxacum mongolicum ethanol extract, a Gerbera jamesonii bolus ethanol extract, a Panax notoginseng ethanol extract, a Monascus purpureus went ethanol extract, a Sanguisorba officinalis ethanol extract, a Ginkgo biloba ethanol extract, a Swertia manshurica ethanol extract, and a Sophora japonica ethanol extract from the Taraxacum mongolicum powder, the Gerbera jamesonii bolus powder, the Panax notoginseng powder, the Monascus purpureus went powder, the Sanguisorba officinalis powder, the Ginkgo biloba powder, the Swertia manshurica powder and the Sophora japonica powder obtained from step (1); (3) the ethanol extracts obtained in step (2), the chitosan, the Camellia oleifera abel and the glycerine are mixed, and then stirred evenly according to the parts by weight to obtain the gel for treating hemorrhoids.

In an embodiment, a preparation method of each ethanol extract in step (2) includes the following steps: the heating reflux extraction is performed on the powder with 90% ethanol for 1 times, followed by recovering a solvent under a reduced pressure to obtain an extract, then the extract is dispersed in a distilled water, and extracted for 2 times with ethyl acetate to obtain the ethanol extract.

In an embodiment, a preparation method of each ethanol extract in step (2) includes the following steps: the heating reflux extraction is performed on the powder with 85% ethanol for 3 times, followed by recovering a solvent under an reduced pressure to obtain an extract, then the extract is dispersed in a distilled water, and extracted for 3 times with the ethyl acetate to obtain the ethanol extract.

### Embodiment 5

A gel for treating hemorrhoids and a preparation method thereof are provided. The gel includes: 40 parts by weight of Taraxacum mongolicum, 40 parts by weight of Gerbera jamesonii bolus, 85 parts by weight of Panax notoginseng, 85 parts by weight of Monascus purpureus went, 20 parts by weight of Sanguisorba officinalis, 20 parts by weight of the chitosan, 10 parts by weight of Camellia oleifera abel, 10 parts by weight of the glycerine, 20 parts by weight of Ginkgo biloba, 20 parts by weight of Swertia manshurica and 20 parts by weight of Sophora japonica.

The preparation method includes the following steps: (1) the Taraxacum mongolicum, the Gerbera jamesonii bolus, the Panax notoginseng, the Monascus purpureus went, the Sanguisorba officinalis, the Ginkgo biloba, the Swertia manshurica, and the Sophora japonica according to the parts by weight are taken to crush thoroughly in a grinder, followed by sieving to obtain a Taraxacum mongolicum powder, a Gerbera jamesonii bolus powder, a Panax notoginseng powder, a Monascus purpureus went powder, a Sanguisorba officinalis powder, a Ginkgo biloba powder, a Swertia manshurica powder, and a Sophora japonica powder, respectively; (2) an ethanol heating reflux extraction is utilized to obtain a Taraxacum mongolicum ethanol extract, a Gerbera jamesonii bolus ethanol extract, a Panax notoginseng ethanol extract, a Monascus purpureus went ethanol extract, a Sanguisorba officinalis ethanol extract, a Ginkgo biloba ethanol extract, a Swertia manshurica ethanol extract, and a Sophora japonica ethanol extract from the Taraxacum mongolicum powder, the Gerbera jamesonii bolus powder, the Panax notoginseng powder, the Monascus purpureus went powder, the Sanguisorba officinalis powder, the Ginkgo biloba powder, the Swertia manshurica powder and the Sophora japonica powder obtained from step (1); (3) the ethanol extracts obtained in step (2), the chitosan, the Camellia oleifera abel and the glycerine are mixed, and then stirred evenly according to the parts by weight to obtain the gel for treating hemorrhoids.

In an embodiment, a preparation method of each ethanol extract in step (2) includes the following steps: the heating reflux extraction is performed on the powder with 90% ethanol for 1 times, followed by recovering a solvent under a reduced pressure to obtain an extract, then the extract is dispersed in a distilled water, and extracted for 2 times with ethyl acetate to obtain the ethanol extract.

In an embodiment, a preparation method of each ethanol extract in step (2) includes the following steps: the heating reflux extraction is performed on the powder with 85% ethanol for 3 times, followed by recovering a solvent under an reduced pressure to obtain an extract, then the extract is dispersed in a distilled water, and extracted for 3 times with the ethyl acetate to obtain the ethanol extract.

### Embodiment 6

A gel for treating hemorrhoids and a preparation method thereof are provided. The gel includes: 70 parts by weight of Taraxacum mongolicum, 70 parts by weight of Gerbera jamesonii bolus, 130 parts by weight of Panax notoginseng, 130 parts by weight of Monascus purpureus went, 40 parts by weight of Sanguisorba officinalis, 30 parts by weight of the chitosan, 20 parts by weight of Camellia oleifera abel, 20 parts by weight of the glycerine, 30 parts by weight of Ginkgo biloba, 35 parts by weight of Swertia manshurica and 35 parts by weight of Sophora japonica.

The preparation method includes the following steps: (1) the Taraxacum mongolicum, the Gerbera jamesonii bolus, the Panax notoginseng, the Monascus purpureus went, the Sanguisorba officinalis, the Ginkgo biloba, the Swertia manshurica, and the Sophora japonica according to the parts by weight are taken to crush thoroughly in a grinder, followed by sieving to obtain a Taraxacum mongolicum powder, a Gerbera jamesonii bolus powder, a Panax notoginseng powder, a Monascus purpureus went powder, a Sanguisorba officinalis powder, a Ginkgo biloba powder, a Swertia manshurica powder, and a Sophora japonica powder, respectively; (2) an ethanol heating reflux extraction is utilized to obtain a Taraxacum mongolicum ethanol extract, a Gerbera jamesonii bolus ethanol extract, a Panax notoginseng ethanol extract, a Monascus purpureus went ethanol extract, a Sanguisorba officinalis ethanol extract, a Ginkgo biloba ethanol extract, a Swertia manshurica ethanol extract, and a Sophora japonica ethanol extract from the Taraxacum mongolicum powder, the Gerbera jamesonii bolus powder, the Panax notoginseng powder, the Monascus purpureus went powder, the Sanguisorba officinalis powder, the Ginkgo biloba powder, the Swertia manshurica powder and the Sophora japonica powder obtained from step (1); (3) the ethanol extracts obtained in step (2), the chitosan, the Camellia oleifera abel and the glycerine are mixed, and then stirred evenly according to the parts by weight to obtain the gel for treating hemorrhoids.

In an embodiment, a preparation method of each ethanol extract in step (2) includes the following steps: the heating reflux extraction is performed on the powder with 90% ethanol for 1 times, followed by recovering a solvent under a reduced pressure to obtain an extract, then the extract is dispersed in a distilled water, and extracted for 2 times with ethyl acetate to obtain the ethanol extract.

In an embodiment, a preparation method of each ethanol extract in step (2) includes the following steps: the heating reflux extraction is performed on the powder with 85% ethanol for 3 times, followed by recovering a solvent under an reduced pressure to obtain an extract, then the extract is dispersed in a distilled water, and extracted for 3 times with the ethyl acetate to obtain the ethanol extract.

### 1. Experimental Materials

(1) Test substance: the gels obtained from embodiments 1-6.
(2) Animals: Japanese big-ear white rabbit with a number of males and a number of females equaled, weighing 1.5-2.5 kilograms (kg), are purchased from Hubei Experimental Animal Research Center;
male Wistar rats, weighing 180-250 grams (g), are purchased from Hubei Experimental Animal Research Center.

### 2. Effect of the Gels Obtained from Embodiments 1-6 on Acetic Acid-Induced Perianal Ulcers in Rabbits

After the Japanese big-ear white rabbits are anesthetized, 20 μL of 36% acetic acid are injected into the submucosa at 4 points distanced 0.5 centimeter (cm) from the anal mouth in the rectum to corrode the rectum, causing ulceration. On the following day, the Japanese big-ear white rabbits are divided into 7 groups according to the degrees of ulcer formation and gender balance, 10 rabbits in each group. The 6 groups are respectively applied with the gels obtained in embodiments 1-6, applying once a day with an amount of applying a dosage of 0.2 grams per kilogram of body weight (0.2 g/kg·bw) for 7 days in a row, that is, 0.5 grams per rabbit, and the 1 group is a blank control group without any applying. The healing of the ulcers is observed and recorded on the eighth day. A therapeutic effect of the gels on the perianal ulcer of rabbits caused by the acetic acid is detected with the ulcer healing degree as an indicator. The results are shown in Table 1 below.

TABLE 1
The therapeutic effect of the gels on perianal
ulcer of rabbits caused by acetic acid

| | Degrees of ulcer | |
|---|---|---|
| Group | Before applying (D 0) | After applying (D 8) |
| Blank control group | 1.6 ± 0.3 | 2.0 ± 0.4 |
| Embodiment 1 | 1.6 ± 0.5 | 4.0 ± 0.7 |
| Embodiment 2 | 1.7 ± 0.2 | 3.6 ± 0.3 |
| Embodiment 3 | 1.6 ± 0.7 | 3.9 ± 0.3 |
| Embodiment 4 | 1.7 ± 0.2 | 2.1 ± 0.5 |
| Embodiment 5 | 1.7 ± 0.3 | 2.6 ± 0.7 |
| Embodiment 6 | 1.6 ± 0.9 | 3.2 ± 0.6 |

It can be seen from Table 1 that the gel can obviously promote the healing of perianal ulcer in rabbits caused by the acetic acid.

### 3. The Effect of Croton Oil on Anal Swelling in the Male Wistar Rats

1 part by weight of distilled water, 4 parts by weight of pyridine, 5 parts by weight of ether, and 10 parts by weight of 6% croton oil ether solution are used to obtain an inflammatory agent. Cotton balls are used to absorb 0.16 mL of the inflammatory agent, followed by inserting each cotton ball to each Wistar rat aged 6 week for ten seconds respectively, so as to establish rat models of anal croton oil swelling. On the following day, according to the degree of swelling, the rats are divided into 7 groups, with 10 in each group. After grouping, the 6 groups are respectively applied with the gels obtained in embodiments 1-6, applying once a day with an amount of applying a dosage of 1 gram per kilogram of body weight (i.e., 0.2 g per rat) for 7 days in a row. The 1 group is a blank control group without any applying. After 30 minutes of the last applying, the rats in each group are euthanized, and 15 mm of rectal tissues from the pubic margin of the anus of rates are cut. After washing with cold physiological saline, the water is absorbed with filter papers. And then five rats are randomly selected from each group, and their tissues are fixed with 10% formaldehyde, embedded in paraffin, sliced, stained with hematoxylin-eosinstaining (HE), and followed by observing for histopathology. The remaining tissues are used to measure the anal swelling in each group of rats. The therapeutic effect of the gels on croton oil induced anal swelling in rats are tested with the results of disease examination and the coefficient of rectal anal swelling as indicators.

TABLE 2
Effect of gels on croton oil induced anal swelling in rats

| Group | Degree of rectal and anal swelling |
|---|---|
| Blank control group | 68.2 ± 3.5 |
| Embodiment 1 | 47.5 ± 3.5 |
| Embodiment 2 | 48.1 ± 4.2 |
| Embodiment 3 | 52.3 ± 5.2 |
| Embodiment 4 | 58.3 ± 5.5 |
| Embodiment 5 | 57.3 ± 4.5 |
| Embodiment 6 | 52.3 ± 3.3 |

It can be seen from Table 2 that the gel can significantly improve the anal swelling and inflammatory reaction of rats caused by croton oil.

### Experiment 2 Safety Testing

### 1. Acute Toxicity Test

After the rabbits fasted for about 16 hours, the gel obtained in embodiment 1 is injected into the rectum with a syringe at the rate of 1 mL/kg per time, and administered respectively in the morning and in the afternoon once. After the administration, the animals are observed daily for any signs of systemic poisoning or death for 14 consecutive days. During the observation period, the appearance, physical signs, behavioral activities, and other general conditions of the rabbits are normal, and the animals do not die. Rabbits are dissected and the rectal administration site and other tissues and organs are observed, and no significant pathological changes are observed. It shows that the gels obtained in embodiments 1-6 have no toxic reaction.

### 2. Skin Irritation Test

The hairs on two sides of the dorsal spine of the rabbits are removed before 24 hours of the experiment, the epidermis of the rabbits must not be not damaged, the gel obtained in embodiment 1 is applied on a side of the skin, followed by covering the skin with two layers of gauze and cellophane, and then fixing the skin with non-irritating adhesive tape and bandage, and the other side is used as a control, and the local reaction of the skin of the rabbit is observed after 24 h, 48 h, and 72 h. After the skin irritation test, no adverse reactions such as erythema and edema are observed at the applying sites after 24 h, 48 h and 72 h. It indicates that the gel for treating hemorrhoids prepared by the disclosure has no skin allergic reaction.

The above is only a preferred embodiment of the disclosure. It should be pointed out that for those skilled in the art, several improvements and embellishments can be made without departing from the principles of the disclosure, and these improvements and embellishments should also be considered as the scope of protection of the disclosure.

What is claimed is:

1. A gel for treating hemorrhoids comprising: Taraxacum mongolicum, Gerbera jamesonii bolus, Panax notoginseng, Monascus purpureus went, Sanguisorba officinalis, chitosan, Camellia oleifera abel, glycerine, Ginkgo biloba, Swertia manshurica and Sophora japonica; and
wherein the Taraxacum mongolicum is 20-80 parts by weight, the Gerbera jamesonii bolus is 20-80 parts by weight, the Panax notoginseng is 50-150 parts by weight, the Monascus purpureus went is 50-150 parts by weight, the Sanguisorba officinalis is 10-50 parts by weight, the chitosan is 10-40 parts by weight, the Camellia oleifera abel is 5-25 parts by weight, the glycerine is 5-25 parts by weight, the Ginkgo biloba is 10-40 parts by weight, the Swertia manshurica is 10-50 parts by weight and the Sophora japonica is 10-50 parts by weight.

2. The gel for treating hemorrhoids as claimed in claim 1, wherein the Taraxacum mongolicum is 20 parts by weight, the Gerbera jamesonii bolus is 20 parts by weight, the Panax notoginseng is 50 parts by weight, the Monascus purpureus went is 50 parts by weight, the Sanguisorba officinalis is 10 parts by weight, the chitosan is 10 parts by weight, the Camellia oleifera abel is 5 parts by weight, the glycerine is 5 parts by weight, the Ginkgo biloba is 10 parts by weight, the Swertia manshurica is 10 parts by weight and the Sophora japonica is 10 parts by weight.

3. The gel for treating hemorrhoids as claimed in claim 1, wherein the Taraxacum mongolicum is 50 parts by weight, the Gerbera jamesonii bolus is 50 parts by weight, the Panax notoginseng is 100 parts by weight, the Monascus purpureus went is 100 parts by weight, the Sanguisorba officinalis is 30 parts by weight, the chitosan is 25 parts by weight, the Camellia oleifera abel is 15 parts by weight, the glycerine is 15 parts by weight, the Ginkgo biloba is 25 parts by weight, the Swertia manshurica is 30 parts by weight and the Sophora japonica is 30 parts by weight.

4. The gel for treating hemorrhoids as claimed in claim 3, wherein a weight ratio of the Taraxacum mongolicum, the Gerbera jamesonii bolus, the Panax notoginseng, the Monascus purpureus went, the Sanguisorba officinalis, the Ginkgo biloba, the Swertia manshurica, and the Sophora japonica is 1:1:1.5-3:1.5-3:0.4-1:0.4-0.8:0.4-1:0.4-1; and a weight ratio of the Taraxacum mongolicum to the chitosan is 1.5-3:1.

5. The gel for treating hemorrhoids as claimed in claim 1, wherein the Taraxacum mongolicum is 80 parts by weight, the Gerbera jamesonii bolus is 80 parts by weight, the Panax notoginseng is 150 parts by weight, the Monascus purpureus went is 150 parts by weight, the Sanguisorba officinalis is 50 parts by weight, the chitosan is 40 parts by weight, the Camellia oleifera abel is 25 parts by weight, the glycerine is 25 parts by weight, the Ginkgo biloba is 40 parts by weight, the Swertia manshurica is 50 parts by weight and the Sophora japonica is 50 parts by weight.

6. The gel for treating hemorrhoids as claimed in claim 1, wherein a weight ratio of the Taraxacum mongolicum and the Gerbera jamesonii bolus is 1:1.

7. The gel for treating hemorrhoids as claimed in claim 1, wherein a weight ratio of the Panax notoginseng and the Monascus purpureus went is 1:1.

8. The gel for treating hemorrhoids as claimed in claim 1, wherein a weight ratio of the Swertia manshurica and the Sophora japonica is 1:1.

9. The gel for treating hemorrhoids as claimed in claim 1, wherein a weight ratio of the Camellia oleifera abel and the glycerine is 1:1.

10. A preparation method of a gel for treating hemorrhoids, wherein the gel for treating hemorrhoids comprises: 20-80 parts by weight of Taraxacum mongolicum, 20-80 parts by weight of Gerbera jamesonii bolus, 50-150 parts by weight of Panax notoginseng, 50-150 parts by weight of Monascus purpureus went, 10-50 parts by weight of Sanguisorba officinalis, 10-40 parts by weight of chitosan, 5-25 parts by weight of Camellia oleifera abel, 5-25 parts by weight of glycerine, 10-40 parts by weight of Ginkgo biloba, 10-50 parts by weight of Swertia manshurica and 10-50 parts by weight of Sophora japonica;
wherein the preparation method comprises the following steps:
(1) taking the Taraxacum mongolicum, the Gerbera jamesonii bolus, the Panax notoginseng, the Monascus purpureus went, the Sanguisorba officinalis, the Ginkgo biloba, the Swertia manshurica, and the Sophora japonica according to the parts by weight; crushing the Taraxacum mongolicum, the Gerbera jamesonii bolus, the Panax notoginseng, the Monascus purpureus went, the Sanguisorba officinalis, the Ginkgo biloba, the Swertia manshurica, and the Sophora japonica in a grinder, followed by sieving to obtain a Taraxacum mongolicum powder, a Gerbera jamesonii bolus powder, a Panax notoginseng powder, a Monascus purpureus went powder, a Sanguisorba officinalis powder, a Ginkgo biloba powder, a Swertia manshurica powder, and a Sophora japonica powder;
(2) utilizing an ethanol heating reflux extraction to obtain a Taraxacum mongolicum ethanol extract, a Gerbera jamesonii bolus ethanol extract, a Panax notoginseng ethanol extract, a Monascus purpureus went ethanol extract, a Sanguisorba officinalis ethanol extract, a Ginkgo biloba ethanol extract, a Swertia manshurica ethanol extract, and a Sophora japonica ethanol extract from the Taraxacum mongolicum powder, the Gerbera jamesonii bolus powder, the Panax notoginseng powder, the Monascus purpureus went powder, the Sanguisorba officinalis powder, the Ginkgo biloba powder, the Swertia manshurica powder and the Sophora japonica powder obtained from step (1); and
(3) mixing the ethanol extracts obtained in step (2), the chitosan, the Camellia oleifera abel and the glycerine, and then stirring evenly to obtain the gel for treating hemorrhoids.

11. The preparation method as claimed in claim 10, wherein the gel comprises: 20 parts by weight of the Taraxacum mongolicum, 20 parts by weight of the Gerbera jamesonii bolus, 50 parts by weight of the Panax notoginseng, 50 parts by weight of the Monascus purpureus went, 10 parts by weight of the Sanguisorba officinalis, 10 parts by weight of the chitosan, 5 parts by weight of the Camellia oleifera abel, 5 parts by weight of the glycerine, 10 parts by weight of the Ginkgo biloba, 10 parts by weight of the Swertia manshurica and 10 parts by weight of the Sophora japonica.

12. The preparation method as claimed in claim 10, wherein the gel comprises: 50 parts by weight of the Taraxacum mongolicum, 50 parts by weight of the Gerbera jamesonii bolus, 100 parts by weight of the Panax notoginseng, 100 parts by weight of the Monascus purpureus went, 30 parts by weight of the Sanguisorba officinalis, 25 parts by weight of the chitosan, 15 parts by weight of the Camellia oleifera abel, 15 parts by weight of the glycerine, 25 parts by weight of the Ginkgo biloba, 30 parts by weight of the Swertia manshurica and 30 parts by weight of the Sophora japonica.

13. The preparation method as claimed in claim 10, wherein the gel comprises: 80 parts by weight of the Taraxacum mongolicum, 80 parts by weight of the Gerbera jamesonii bolus, 150 parts by weight of the Panax notoginseng, 150 parts by weight of the Monascus purpureus went, 50 parts by weight of the Sanguisorba officinalis, 40 parts by weight of the chitosan, 25 parts by weight of the Camellia oleifera abel, 25 parts by weight of the glycerine, 40 parts by weight of the Ginkgo biloba, 50 parts by weight of the Swertia manshurica and 50 parts by weight of the Sophora japonica.

14. The preparation method as claimed in claim 10, wherein a weight ratio of the Taraxacum mongolicum, the Gerbera jamesonii bolus, the Panax notoginseng, the Monascus purpureus went, the Sanguisorba officinalis, the Ginkgo biloba, the Swertia manshurica, and the Sophora japonica is 1:1:1.5-3:1.5-3:0.4-1:0.4-0.8:0.4-1:0.4-1; and a weight ratio of the Taraxacum mongolicum to the chitosan is 1.5-3:1.

15. The preparation method as claimed in claim 10, wherein the step (2) comprises: performing the following steps for each of the Taraxacum mongolicum powder, the Gerbera jamesonii bolus powder, the Panax notoginseng powder, the Monascus purpureus went powder, the Sanguisorba officinalis powder, the Ginkgo biloba powder, the Swertia manshurica powder and the Sophora japonica powder:
performing heating reflux extraction on the powder with 85-90% ethanol for 1-3 times, followed by recovering a solvent under a reduced pressure to obtain an extract, then dispersing the extract in a distilled water, and extracting 2-4 times with ethyl acetate to obtain the ethanol extract.

16. The preparation method as claimed in claim 15, wherein the step (2) comprises: performing the following steps for each of the Taraxacum mongolicum powder, the Gerbera jamesonii bolus powder, the Panax notoginseng powder, the Monascus purpureus went powder, the Sanguisorba officinalis powder, the Ginkgo biloba powder, the Swertia manshurica powder and the Sophora japonica powder:
performing heating reflux extraction on the powder with 85% ethanol for 3 times, followed by recovering the solvent under the reduced pressure to obtain the extract, then dispersing the extract in the distilled water, and extracting 3 times with the ethyl acetate to obtain the ethanol extract.

17. The preparation method as claimed in claim 11, wherein the step (2) comprises performing the following steps for each of the Taraxacum mongolicum powder, the Gerbera jamesonii bolus powder, the Panax notoginseng powder, the Monascus purpureus went powder, the Sanguisorba officinalis powder, the Ginkgo biloba powder, the Swertia manshurica powder and the Sophora japonica powder:
performing heating reflux extraction on the powder with 90% ethanol, followed by recovering a solvent under a reduced pressure to obtain an extract, then dispersing the extract in a distilled water, and extracting 2 times with ethyl acetate to obtain the ethanol extract.

18. The preparation method as claimed in claim 12, wherein the step (2) comprises performing the following steps for each of the Taraxacum mongolicum powder, the Gerbera jamesonii bolus powder, the Panax notoginseng powder, the Monascus purpureus went powder, the Sanguisorba officinalis powder, the Ginkgo biloba powder, the Swertia manshurica powder and the Sophora japonica powder:
performing heating reflux extraction on the powder with 85% ethanol for 2 times, followed by recovering a solvent under a reduced pressure to obtain an extract, then dispersing the extract in a distilled water, and extracting 3 times with ethyl acetate to obtain the ethanol extract.

19. The preparation method as claimed in claim 13, wherein the step (2) comprises: performing the following steps for each of the Taraxacum mongolicum powder, the Gerbera jamesonii bolus powder, the Panax notoginseng powder, the Monascus purpureus went powder, the Sanguisorba officinalis powder, the Ginkgo biloba powder, the Swertia manshurica powder and the Sophora japonica powder:
performing heating reflux extraction on the powder with 90% ethanol for 3 times, followed by recovering a solvent under a reduced pressure to obtain an extract, then dispersing the extract in a distilled water, and extracting 4 times with ethyl acetate to obtain the ethanol extract.

20. A preparation method of a gel for treating hemorrhoids comprising the following steps:
(1) crushing 20 parts by weight of Taraxacum mongolicum, 20 parts by weight of Gerbera jamesonii bolus, 50 parts by weight of Panax notoginseng, 50 parts by weight of Monascus purpureus went, 10 parts by weight of Sanguisorba officinalis, 10 parts by weight of Ginkgo biloba, 10 parts by weight of Swertia manshurica, and 10 parts by weight of Sophora japonica individually, and then sieving to obtain a Taraxacum mongolicum powder, a Gerbera jamesonii bolus powder, a Panax notoginseng powder, a Monascus purpureus went powder, a Sanguisorba officinalis powder, a Ginkgo biloba powder, a Swertia manshurica powder, and a Sophora japonica powder;
(2) performing ethanol heating reflux extraction on each of the Taraxacum mongolicum powder, the Gerbera jamesonii bolus powder, the Panax notoginseng powder, the Monascus purpureus went powder, the Sanguisorba officinalis powder, the Ginkgo biloba powder, the Swertia manshurica powder and the Sophora japonica powder to obtain a Taraxacum mongolicum ethanol extract, a Gerbera jamesonii bolus ethanol extract, a Panax notoginseng ethanol extract, a Monascus purpureus went ethanol extract, a Sanguisorba officinalis ethanol extract, a Ginkgo biloba ethanol extract, a Swertia manshurica ethanol extract, and a Sophora japonica ethanol extract; and
(3) mixing the ethanol extracts obtained in step (2), 10 parts by weight of chitosan, 5 parts by weight of Camellia oleifera abel and 5 parts by weight of glycerine, and then stirring evenly to obtain the gel for treating hemorrhoids.
